(19) United States
(12) Patent Application Publication
(10) Pub. No.: US 2016/0081566 A1
(43) Pub. Date: Mar. 24, 2016

(54) IDENTIFYING A TYPE OF CARDIAC EVENT FROM A CARDIAC SIGNAL SEGMENT

(71) Applicant: Xerox Corporation, Norwalk, CT (US)

(72) Inventors: Beilei XU, Penfield, NY (US); Survi KYAL, Rochester, NY (US); Lalit Keshav MESTHA, Fairport, NY (US)

(21) Appl. No.: 14/492,948

(22) Filed: Sep. 22, 2014

(51) Int. Cl. A61B 5/024; A61B 5/00; A61B 5/11; A61B 5/0476; A61B 8/02; A61B 5/0464; A61B 7/02; A61B 5/04; A61B 5/044; A61B 5/02; A61B 5/0456; A61B 5/046; A61B 5/0245; A61B 5/0488

(52) U.S. Cl. CPC A61B 5/02405; A61B 5/0245; A61B 5/7264; A61B 5/1102; A61B 5/0476; A61B 8/02; A61B 5/0488; A61B 7/02; A61B 5/02416; A61B 5/04012; A61B 5/746; A61B 5/747; A61B 5/044; A61B 5/6898; A61B 5/0006; A61B 5/02028; A61B 5/0456; A61B 5/046; A61B 5/0464; A61B 2560/0475

(57) ABSTRACT

What is disclosed is a system and method identifying a type of cardiac event from cardiac signals obtained from a subject. In one embodiment, at least two clusters are formed. Each cluster is associated with a different cardiac event based on features of interest identified from various cardiac signal segments. At least one of the clusters is associated with a cardiac event which is an arrhythmia and one of the clusters is associated with a non-arrhythmia. A new cardiac signal segment of a subject is received. The signal segment is analyzed to identify features of interest. A distance is calculated between each of the clusters and the identified features of interest obtained from having analyzed the subject's cardiac signal segment. A cardiac event is identified for the subject based on the type of cardiac event associated with the cluster which the subject's features of interest had a shortest distance to.

### TECHNICAL FIELD

The present invention is directed to systems and methods for identifying a type of cardiac event from a cardiac signal segment obtained from a subject being monitored for cardiac function assessment.

### BACKGROUND

Early detection of cardiac arrhythmias can be critical for patient recovery. Increasingly sophisticated systems and methods for monitoring for various cardiac events are needed to improve diagnosis and treatment. The present invention is directed toward identifying a cardiac event from a cardiac signal segment obtained from a subject being monitored for cardiac function assessment.

### INCORPORATED REFERENCES

The following U.S. patents, U.S. patent applications, and Publications are incorporated herein in their entirety by reference.

“Estimating Cardiac Pulse Recovery From Multi-Channel Source Data Via Constrained Source Separation”, U.S. patent application Ser. No. 13/247,683, by Mestha et al.

“Video-Based Estimation Of Heart Rate Variability”, U.S. patent application Ser. No. 13/532,057, by Mestha et al.

“Systems And Methods For Non-Contact Heart Rate Sensing”, U.S. patent application Ser. No. 13/247,575, by Mestha et al.

“Continuous Cardiac Pulse Rate Estimation From Multi-Channel Source Video Data”, U.S. patent application Ser. No. 13/528,307, by Kyal et al.

“Continuous Cardiac Pulse Rate Estimation From Multi-Channel Source Video Data With Mid-Point Stitching”, U.S. patent application Ser. No. 13/871,728, by Kyal et al.

“Continuous Cardiac Signal Generation From A Video Of A Subject Being Monitored For Cardiac Function”, U.S. patent application Ser. No. 13/871,766, by Kyal et al.

“Determining Cardiac Arrhythmia From A Video Of A Subject Being Monitored For Cardiac Function”, U.S. patent application Ser. No. 13/532,128, by Mestha et al.

### BRIEF SUMMARY

What is disclosed is a system and method for identifying a type of cardiac event from a cardiac signal segment obtained from a subject being monitored for cardiac function. One embodiment of the present method involves forming at least two clusters each associated with a different cardiac event. Elements of the clusters comprise cardiac signal segments which have been either manually or automatically assigned to the clusters based on features of interest obtained from the cardiac signal segments. At least one of the clusters is associated with a cardiac event which is an arrhythmic event such as, for instance, atrial fibrillation, ventricular premature contraction, ventricular tachycardia, sinus bradycardia, or sinus tachycardia. Once the clusters have been formed, a cardiac signal segment of a subject being monitored for a cardiac event is received. The new cardiac signal segment is analyzed to determine at least one feature of interest. In a manner more fully disclosed herein, a type of cardiac event is identified for the subject based on a type of cardiac event associated with a cluster which the feature of interest obtained from the subject's cardiac signal segment had a shortest distance in relation to either a center of the cluster, a boundary element of the cluster, or a weighted sum of elements in that cluster. Many features and advantages of the present method will become readily apparent from the following detailed description and accompanying drawings.

### BRIEF DESCRIPTION OF THE DRAWINGS

The foregoing and other features and advantages of the subject matter disclosed herein will be made apparent from the following detailed description taken in conjunction with the accompanying drawings, in which:

FIG. 1 shows a schematic diagram of normal sinus rhythm for a human heart as seen on an electrocardiogram (ECG);

FIG. 2 shows a video image device capturing video of a subject;

FIG. 3 shows the subject of FIG. 2 laying in an upright position with a plurality of contact-based electrodes attached to the chest where cardiac signals are actively being captured by an electrocardiographic device;

FIG. 4 shows a plurality of clusters each associated with a different type of cardiac event;

FIG. 5 is a flow diagram which illustrates one example embodiment of the present method for identifying a type of cardiac event from a cardiac signal segment obtained from the subject of FIGS. 2 and 3;

FIG. 6 is a continuation of the flow diagram of FIG. 5 with flow processing continuing with respect to node A;

FIG. 7 illustrates a block diagram of one example signal processing system for identifying a type of cardiac event from a cardiac signal segment obtained from the subject in accordance with the embodiment described with respect to the flow diagrams of FIGS. 5 and 6;

FIG. 8 plots two metrics, i.e., PHS and pNN50, calculated before/after the patient went through a cardioversion procedure;

FIG. 9 shows an example using a log-transform of our raw data used for performance testing of the methods disclosed herein;

FIG. 10 shows a bi-modal Gaussian-Mixture Model (GMM) estimated from the data set of FIGS. 8 and 9;

FIGS. 11 and 12 plot the Mahalanobis distance from SR and AF data points, respectively, to cluster centers;

FIGS. 13 and 14 plot PHS vs. pNN50 for 2 patients who went through a cardioversion procedure; and

FIG. 15 shows the classification results for 11 patients using a trained SVM classifier in a LOOCV (i.e., the leave one out cross-validation) experiment.

### DETAILED DESCRIPTION

What is disclosed is a system and method for identifying a type of cardiac event from a cardiac signal segment obtained from a subject being monitored for cardiac function assessment.

### Non-Limiting Definitions

A “subject” refers to a living being. Although the term “person” or “patient” may be used throughout this disclosure, it should be appreciated that the subject may be something other than a human such as, for example, a primate. Therefore, the use of such terms is not to be viewed as limiting the scope of the appended claims strictly to humans.

A “cardiac event” is an event associated with the function of the heart. Cardiac events can be arrhythmic and/or non-arrhythmic.

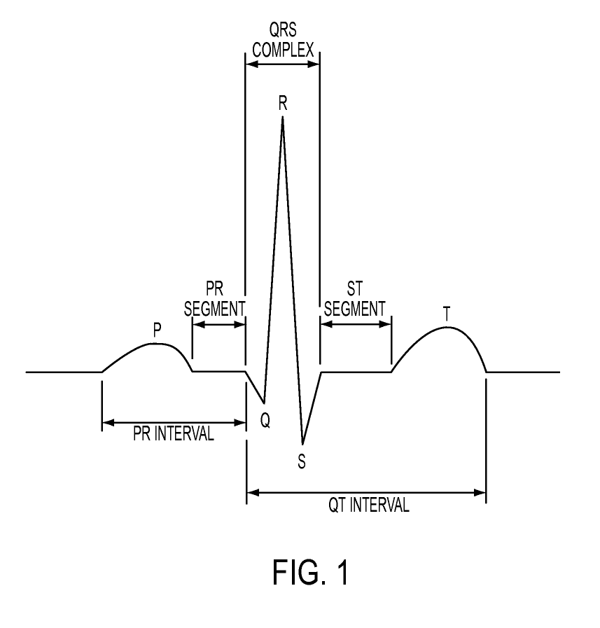

“Cardiac arrhythmia”, (also known as cardiac dysrhythmia), refers to an irregular heartbeat. One common cardiac arrhythmia is atrial fibrillation. Other arrhythmias include ventricular tachycardia, sinus tachycardia, sinus bradycardia, and ventricular premature contraction, as are generally understood in the medical arts. A non-arrhythmic refers to the heart's normal sinus rhythm. FIG. 1 shows a schematic diagram of sinus rhythm (SR) for a human heart as seen on an electrocardiogram. Although a brief description of various arrhythmias is provided, the reader is referred to any of a variety of medical literature and textbooks for a more complete discussion.

“Atrial fibrillation” (AF or A-fib) is one of the most common cardiac arrhythmias. In AF, electrical impulses generated by the sinoatrial node are overwhelmed by disorganized electrical impulses leading to irregular conduction of impulses to the ventricles which generate the heartbeat. In AF, the P-waves are absent with unorganized electrical activity in their place with irregular RR intervals due to irregular conduction of impulses to the ventricles (which may be difficult to determine if the rate is rapid). AF may occur in episodes lasting from minutes to days, or be permanent in nature. A number of medical conditions increase the risk of AF including a narrowing of the orifice of the mitral valve (“mitral stenosis”).

“Ventricular tachycardia” is an arrhythmia that is tachycardia (rapid heart rate) originating in the ventricles of the heart. Ventricular tachycardia can lead to ventricular fibrillation, asystole, and heart failure.

“Sinus tachycardia” is an arrhythmia that is tachycardia due to an elevated rate of impulses originating in the sinoatrial node. In humans, sinus tachycardia is often defined as a heart rate that is greater than 100 bpm.

“Sinus bradycardia” is an arrhythmia that is bradycardia (slow heart rate) originating from the sinoatrial node. In humans, sinus bradycardia is often defined as a heart rate that is less than 60 bpm.

“Ventricular premature contraction” (VPC), also known as premature ventricular contraction (PVC), ventricular premature beat (VPB), and ventricular extrasystole (VES), occurs when the heartbeat is being initiated by Purkinje fibers in the ventricles rather than by the conductive activity of the sinoatrial node. VPC arrhythmias are often benign. Single beat VPCs can be asymptomatic in relatively healthy individuals.

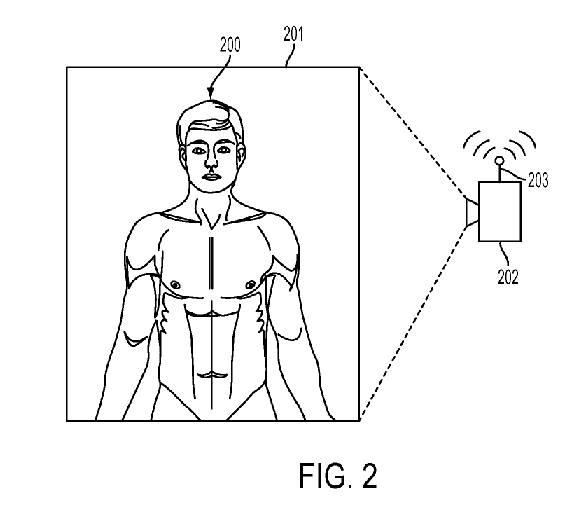

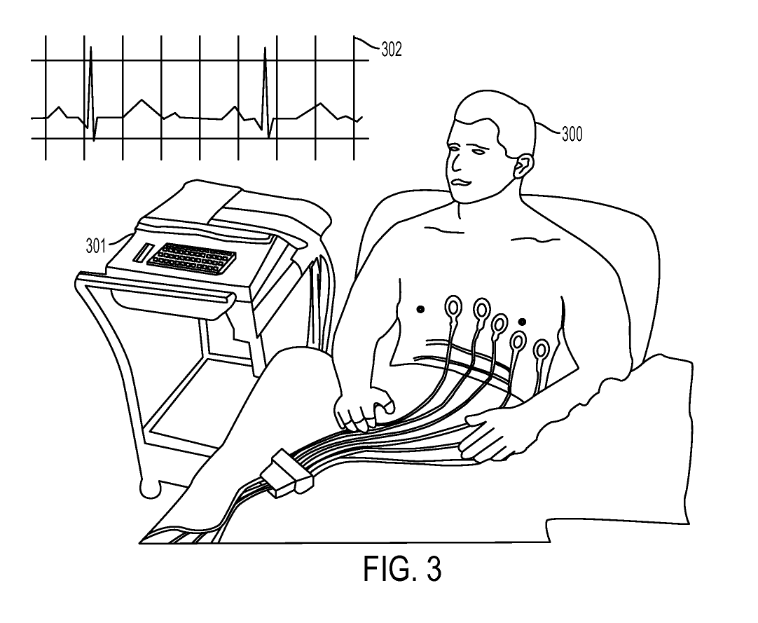

A “cardiac signal” is a signal which relates to the function of the subject's heart. Cardiac signals can be, for instance, a videoplethysmographic (VPG) signal extracted from a time-series signal obtained from processing overlapping batches of image frames captured by an imaging device such as, a video camera, a RGB camera, a multi-spectral or hyperspectral imaging system, and a hybrid device comprising any combination thereof. Such imaging devices typically have a plurality of outputs where the captured images are obtained on a per-channel basis. FIG. 2 shows one example embodiment of a video imaging device 202 actively capturing video 201 of a subject 200. The video image frames of the subject are communicated to a remote computing device via a wireless transmissive element 203, shown as an antenna, where the image frames of the video are processed in partially overlapping batches to obtain a time-series signal. VPG signals are extracted from the time-series signals. Methods for obtaining a time-series signal from video image frames and for extracting VPG signals are disclosed in several of the incorporated references. A cardiac signal can also be received from specialized medical instrumentation such as, for instance, an electrocardiographic device, an echocardiographic device, an electromyographic device, an electroencephalographic device, a phonocardiographic device, and a ballistocardiographic device. FIG. 3 shows a patient 300 laying in an upright position with a plurality of contact-based electrodes attached to the chest where cardiac signals are actively being captured by an electrocardiographic device 301 on a cart. The electrocardiographic device receives cardiac signals from the patient. In one embodiment, the patient's cardiac signal takes the form of an electrocardiogram 302. Various imaging devices and specialized medical instrumentation for obtaining cardiac signals may incorporate memory, a storage device, and one or more microprocessors executing machine readable program instructions.

A “cardiac signal segment” is at least a portion of a cardiac signal. A cardiac signal segment can be of any length. Methods for obtaining a segment of a signal are well established in the signal processing arts. The cardiac signal segment can be normalized to a frequency of a normalized heartbeat. A length of the cardiac signal segment can be a single cardiac cycle or a normalized cardiac cycle. Cardiac signal segments are analyzed to obtain features of interest.

“Features of interest” are obtained by analyzing a cardiac signal segment and are used herein for distance determination and cardiac event identification. A feature of interest may be one or more aspects of, for instance, a frequency domain version of the cardiac signal segment. Features of interest may take the form of one or more higher order statistical quantities obtained from analyzing the cardiac signal segment. Features of interest may also take the form of one or more heart rate variability metrics obtained from analyzing the cardiac signal segment. Patient information and medical histories may further be associated with various cardiac signal segments and features of interest.

A “higher order statistical quantity” is obtained by analyzing a set of peak-to-peak intervals of a cardiac signal segment with respect to any of: a mean, standard deviation, skewness, and kurtosis.

A “heart rate variability metric” is obtained by analyzing a cardiac signal segment with respect to any of: a Standard Deviation of RR Intervals (SDRR), Root Mean Square of Successive RR Difference (RMSSD), Proportion of NN or RR interval exceeding 50 milliseconds (pNN50), Shannon Entropy (ShE), Standard Deviation 1 (SD1), Standard Deviation 2 (SD2), Pulse Harmonic Strength (PHS), and Normalized Pulse Harmonic Strength (NPHS).

A “fundamental frequency” (or simply the “fundamental”) is the frequency of a periodic waveform with the highest power. The fundamental is given by:

$${f}_{0} = \frac{1}{T}$$

where T is the fundamental period. The first harmonic is often abbreviated as f₁. In some contexts, the fundamental f₀ is the first harmonic. If the fundamental frequency is f₀, the harmonics have frequencies 2f₀, 3f₀, 4f₀, . . . , etc. Harmonics have the property that they are all periodic at the fundamental. Therefore, the sum of the harmonics is also periodic at the fundamental frequency.

“Power spectral density” (PSD), describes how the power of a signal or time series is distributed over different frequencies contained within that signal. In general, the power P of a signal x(t) is an average over the time interval [−T,T], as given by:

$$P = {lim}_{T \to ∞} \frac{1}{2 T} {\int }_{- T}^{T} {x ( t )}^{2} d t$$

It is advantageous to work with a truncated Fourier transform where the signal is integrated only over a finite interval. Methods for computing power spectral densities are well understood in the signal processing arts. From the PSD, the fundamental frequency and its harmonics are identified.

“Pulse Harmonic Strength (PHS)” is a ratio of signal strength at the fundamental frequency and harmonics to a strength of a base signal without these fundamental frequency and harmonics. Frequencies in a neighborhood of these harmonics defines a band. In the present context, this band is around 0.2 Hz bpm). PHS represents the total strength of the pulse power because the power is centered at heartbeats and the harmonics of those beats. All the power is integrated within this band, denoted P_{sig}. Power in all remaining bands is integrated separately, denoted Pₙₒᵢ. PHS is given by:

$$PHS = {P}_{sig} / {P}_{noi}$$

$${P}_{noi} = {P}_{total} - {P}_{sig} .$$

where P_{Total} is the total energy of the signal.

“Normalized Pulse Harmonic Strength (NPHS)” is a ratio of signal strength at the fundamental frequency and harmonics to a strength of a base signal. The NPHS has a value between 0 and 1 is given by:

$$NPHS = {P}_{sig} / {P}_{Total}$$

A “cluster” contains cardiac signal segments which are associated with the same type of cardiac event based on features of interest determined for each of the signal segments. At least one of the clusters is associated with a cardiac event which is an arrhythmia. In various embodiments, one of the clusters is associated with a cardiac event which is a non-arrhythmic event. Methods for forming clusters based on features obtained from analyzing signal segments include K-means testing, vector quantization (such as the Linde-Buzo-Gray algorithm), constrained clustering, fuzzy clustering, nearest neighbor clustering, linear discriminant analysis, Gaussian Mixture Model, and a support vector machine, as are generally understood. Various thresholds may be employed to facilitate further discrimination amongst sets of features. Clusters may be labeled based on electrocardiographic traces, apriori knowledge of different types of cardiac events, and a heart rate variability metric. Clusters may also be formed manually and/or labeled manually. The clusters are formed in advance of analyzing a subject's cardiac signal segment for features of interest. The clusters are used to facilitate an identification of a cardiac event for the subject.

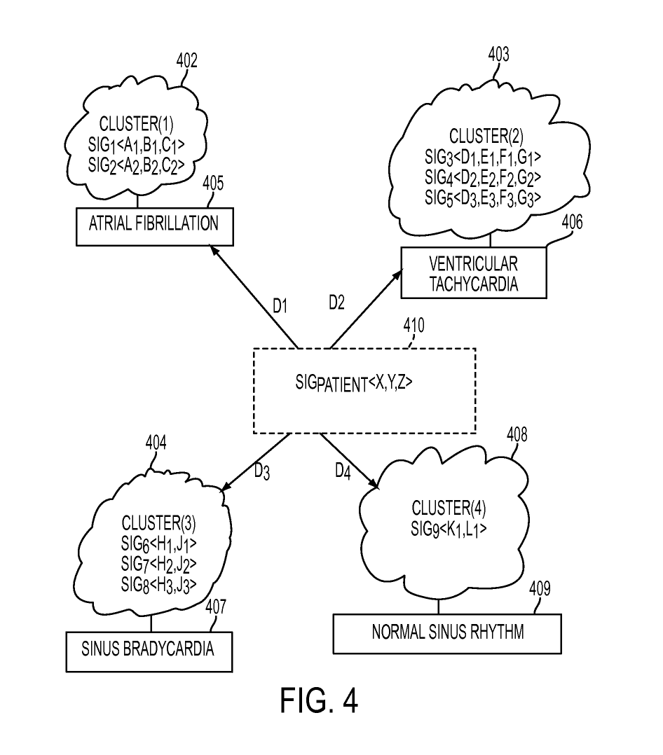

Reference is now being made to the embodiment of FIG. 4 which shows a plurality of clusters each associated with a different type of cardiac event. A first cluster, shown as cluster(1) at 402, is shown comprising two cardiac signal segments, denoted SIG₁ and SIG₂. The cardiac event 405 associated with cluster 402 is Atrial Fibrillation. Features of interest are obtained from having analyzed the two signal segments of cluster 402. These features help define which cardiac event a given signal segment is to be assigned. In the embodiment shown, the features of interest obtained from having analyzed the first and second cardiac signal segments are given by: SIG₁<A₁,B₁,C₁> and SIG₂<A₂,B₂,C₂>, respectively, where <A,B,C> represent different features that facilitated the association of SIG₁ and SIG₂ with the cardiac event of Atrial Fibrillation. Likewise, a second cluster, shown as cluster(2) at 403, comprises three cardiac signal segments, denoted SIG₃, SIG₄ and SIG₅. The cardiac event 406 associated with cluster 403 is Ventricular Tachycardia. Features of interest obtained from having analyzed the cardiac signal segments of cluster(2) are given by: SIG₃<D₁,E₁,F₁>, SIG₄<D₂,E₂,F₂> and SIG₅<D₃,E₃,F₃>, respectively, where <D,E,F> represent features that these three signal segments have in common. Features <D,E,F> facilitated the association of SIG₃, SIG₄ and SIG₅ with the cardiac event of Ventricular Tachycardia. A third cluster, shown as cluster(3) at 404, also comprises three cardiac signal segments, denoted SIG₆, SIG₇ and SIG₈. The cardiac event 407 associated with cluster 404 is Sinus Bradycardia. Features of interest obtained from having analyzed the cardiac signal segments of cluster(3) are given by: SIG₆<H₁,J₁,>, SIG₇<H₂,J₂,>, and SIG₈<H₃,J₃,>, respectively, where <H,J> represent features that these three signal segments have in common. Features <H,J> facilitated the association of SIG₆, SIG₇ and SIG₈ with the cardiac event of Sinus Bradycardia. A fourth cluster, shown as cluster(4) at 408, is associated with the cardiac event 409 of Normal Sinus Rhythm, (i.e., a non-arrhythmic event). Cluster(4) comprises one cardiac signal segment, denote SIG₉. Features of interest obtained from having analyzed the cardiac signal segment of Cluster(4) at 408 are given by: SIG₉<K₁,L₁,>, where features <K,L> facilitated the association of SIG₉ with Normal Sinus Rhythm. Also shown at insert 410, is a new cardiac signal segment, denoted SIG_{Patient}, which has been received for processing. Features of interest obtained from having analyzed the patient's cardiac signal segment are given by: SIGₚₐₜᵢₑₙₜ<X,Y,Z>. In accordance with the teachings hereof, a determination is to be made as to which cluster the features of interest obtained from the patient's cardiac signal segment have a minimum distance of either D₁, D₂, D₃, or D₄ such that a cardiac event can be identified for the subject.

“Identifying a cardiac event” for a subject means to determine which cluster the feature(s) of interest obtained from the subject's cardiac signal segment had a shortest distance in relation to either a center of the cluster, a boundary element of the cluster, or a weighted sum of one or more elements comprising the cluster. Given Cluster(1 . . . n) where n≧2, the identified cardiac event is determined by:

Min(D₁,D₂, . . . ,Dₙ)

Event←Cluster(j)

where Dᵢ is the distance between the i^{th} cluster and the feature of interest obtained from analyzing the subject's cardiac signal segment. The identified event is the cardiac event associated with the j^{th} cluster with a minimum distance Dⱼ. In the example of FIG. 4, assume that the D₃ was the minimum distance. As such, the cardiac event identified for the subject based on the features of interest obtained from having analyzed the subject's cardiac signal segment would be the event which is associated with cluster(3) at 404, i.e., Sinus Bradycardia 407. Likewise, if D₄ was the minimum distance then the cardiac event identified for the subject based on the features of interest obtained from having analyzed the subject's cardiac signal segment would be the event which is associated with cluster(4) at 408, i.e., Normal Sinus Rhythm 409.

The steps of “identifying”, “analyzing”, “obtaining” and “processing”, as used herein, include the application of various signal processing and mathematical operations applied to signals, according to any specific context or for any specific purpose. The terms in the Detailed Description and claims hereof are intended to include any activity, in hardware or software, having the substantial effect of the mathematical or signal-processing action (e.g. subtracting, averaging, detrending). It should be appreciated that such steps may be facilitated or otherwise effectuated by a microprocessor executing machine readable program instructions retrieved from a memory or storage device.

### Flow Diagram of One Embodiment

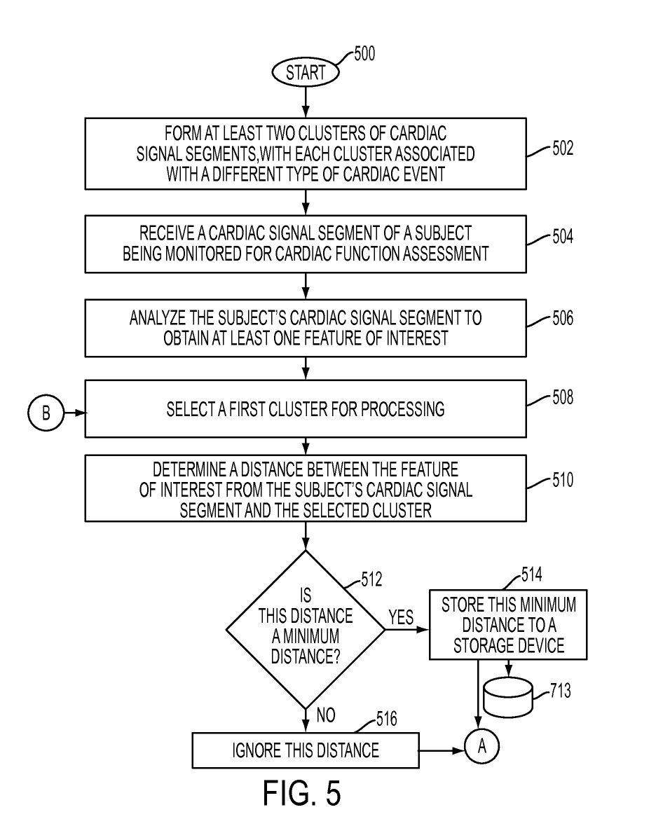

Reference is now being made to the flow diagram of FIG. 5 which illustrates one example embodiment of the present method for identifying a type of cardiac event from a cardiac signal segment obtained from a subject. Flow processing begins at step 500 and immediately proceeds to step 502.

At step 502, form at least two clusters of cardiac signal segments with each cluster associated with a different type of cardiac event. Elements of the clusters have been assigned to the clusters based on features of interest obtained from their respective cardiac signal segments. Example clusters are shown and discussed with respect to FIG. 4.

At step 504, receive a cardiac signal segment of a subject being monitored for cardiac function assessment. The subject's cardiac signal segment has not yet been assigned to one of the clusters (formed in step 502).

At step 506, analyze the subject's cardiac signal segment to obtain at least one feature of interest. The subject's cardiac signal segment can be analyzed with respect to any of the features of interest disclosed herein.

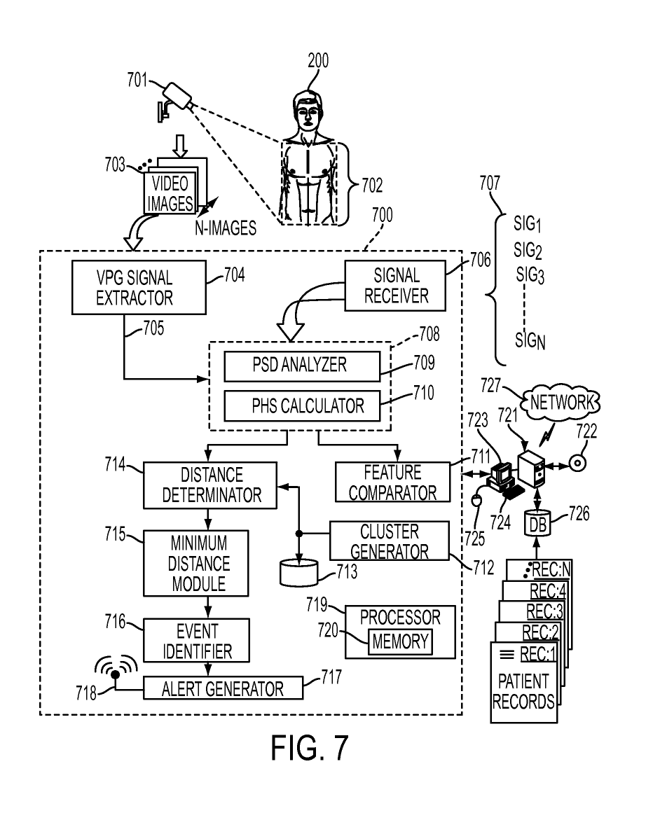

At step 508, select a first of the clusters (formed in step 502). A selection can be effectuated using the user interface of the workstation of FIG. 7.

At step 510, determine a distance between the feature of interest from the subject's cardiac signal segment and the selected cluster. The distance can be in relation to either a center of the cluster, a boundary element of the cluster, or a weighted sum of one or more elements comprising the cluster.

At step 512, a determination is made whether this distance is a minimum distance. If so then, at step 514, store this minimum distance to a memory or storage device 713 and proceed to node A. The cluster associated with this minimum distance is also stored. Otherwise, if this distance is not a minimum distance then, at step 516, ignore this distance and proceed to node A.

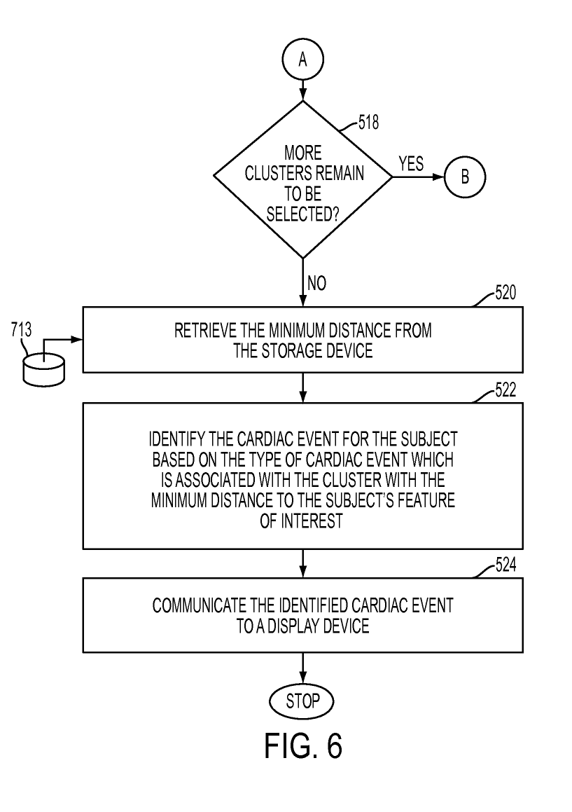

Reference is now being made to the flow diagram of FIG. 6 which is a continuation of the flow diagram of FIG. 5 with flow processing continuing with respect to node A.

At step 518, a determination is made whether more clusters remain to be selected. If so then processing continues with respect to node B wherein, at step 508, a next cluster is selected. A distance is then determined between the feature of interest from the subject's cardiac signal segment and this next selected cluster. A determination is then made whether this next distance is a minimum distance. If so then this new minimum distance and the associated cluster are stored (at step 514) to replace the previously stored data. Flow processing repeats in a similar manner until, at step 508, no more clusters remain to be selected. Thereafter, flow processing continues with respect to step 520.

At step 520, retrieve the minimum distance and the cluster associated with this minimum distance from the storage device 713.

At step 522, identify the cardiac event for the subject based on the type of cardiac event which is associated with the cluster with the minimum distance to the subject's feature of interest.

At step 524, communicate the identified cardiac event to a display device. In this embodiment, further processing stops. In other embodiments, the identified cardiac event is communicated to a memory, a storage device, a handheld wireless device, a handheld cellular device, and a remote device over a network. An alert signal may be initiated and a signal may further be sent to a medical professional.

It should be appreciated that the flow diagrams depicted herein are illustrative. One or more of the operations illustrated in the flow diagrams may be performed in a differing order. Other operations may be added, modified, enhanced, or consolidated. Variations thereof are intended to fall within the scope of the appended claims.

### Block Diagram of Signal Processing System

Reference is now being made to FIG. 7 which illustrates a block diagram of one example signal processing system 700 for identifying a type of cardiac event from a cardiac signal segment obtained from the subject in accordance with the embodiment described with respect to the flow diagrams of FIGS. 5 and 6.

Video imaging device 701 acquires streaming video of an exposed body region 702 of the subject 200 being monitored for cardiac function assessment in accordance with the teachings hereof. Video image frames (collectively at 703) are communicated to a VPG Signal Extractor 704 which receives batches of image frames and isolates pixels associated with the exposed body region in each of the image frames. The isolated pixels are processed to obtain a time-series signal for each batch. A VPG signal is then extracted from the time-series signal in a manner as disclosed in several of the incorporated references. VPG Signal Extractor 704 outputs a cardiac signal segment 705 comprising, in this embodiment, a VPG signal corresponding to the subject's cardiac function.

In another stage, Signal Receiver 706 receives a total of n≧2 cardiac signal segments (collectively at 707) and provides the received cardiac signal segments to a Feature Extractor Module 708 comprising, in this embodiment, a PSD Analyzer 709 and a PHS Calculator 710. The PSD Analyzer 709 computes a power spectral density across all frequencies within the cardiac signal segments. PHS Calculator 710 calculates a pulse harmonic strength for each received cardiac signal segment. Comparator 711 functions to compare the results of the PSD Analyzer and the PHS Calculator to parameters which are known to be associated with different types of cardiac events. A result of each comparison is provided to Cluster Generator 712 which proceeds to assign each of the received cardiac signal segments 707 to a cluster. New clusters are formed as needed. In such a manner, clusters associated with different cardiac events are formed and stored to storage device 713. An example of formed clusters and various assigned cardiac signal segments is shown and discussed with respect to FIG. 4.

After the clusters have been formed and all the received cardiac signal segments 707 have been assigned to respective clusters, in a next stage the VPG signal 705 corresponding to the subject's cardiac function is received by the Feature Extractor Module 708 wherein the PSD Analyzer 709 and the PHS Calculator 710 perform their functionality on the subject's cardiac signal segment 705 such that features of interest can be obtained therefrom. Example features of interest obtained from processing the subject's cardiac signal segment (VPG Signal) are shown in FIG. 4 at 410. The extracted features of interest are provided to Distance Determinator 714. Determinator 714 retrieves the generated clusters and the elements thereof from storage device 703 and proceeds to determine a distance between the features of interest obtained from analyzing the subject's cardiac signal segment 705 and each of the formed clusters generated by Cluster Generator 712. Distance Determinator 714 provides the calculated distances to a Minimum Distance Module 715 which functions to determine which of the calculated distances was a minimum distance. A result thereof is provided to Event Identifier 716 which proceeds to identify the type of cardiac event associated with the cluster having a minimum distance to the features of interest obtained from the subject's signal segment 705. Alert Generator 717 receives the identified cardiac event and, in response to the cardiac event being an arrhythmia, proceeds to initiate an alert signal to a display device via antenna 718.

Central Processor (CPU) 719 retrieves machine readable program instructions from Memory 720 and is provided to facilitate the functionality of any of the modules of the signal processing system 700. The processor 719, operating alone or in conjunction with other processors, may be configured to assist or otherwise perform the functionality of any of the block modules of system 700. Processor 719 further facilitates communication between system 700 workstation 721.

Workstation 721 has a computer case which houses various components such as a motherboard with a processor and memory, a network card, a video card, a hard drive capable of reading/writing to machine readable media 722 such as a floppy disk, optical disk, CD-ROM, DVD, magnetic tape, and the like, and other software and hardware needed to perform the functionality of a computer workstation. The workstation further includes a display device 723, such as a CRT, LCD, or touchscreen device, for displaying information, video, distances, clusters, features of interest, computed values, medical information, results, and the like, which are produced or are otherwise generated by any of the block modules of system 700. A user can view any of that information and make a selection from menu options displayed thereon. Keyboard 724 and mouse 725 effectuate a user input or selection as needed.

The workstation implements a database in storage device 726 wherein patient records are stored, manipulated, and retrieved in response to a query. Such records, in various embodiments, take the form of patient medical history stored in association with information identifying the patient along with medical information. It should be appreciated that database 726 may be the same as storage device 713 or, if separate devices, may contain some or all of the information contained in either storage device. Although the database is shown as an external device, the database may be internal to the workstation mounted, for example, on a hard disk therein. It should be appreciated that the workstation has an operating system and other specialized software configured to display alphanumeric values, menus, scroll bars, dials, slideable bars, pull-down options, selectable buttons, and the like, for entering, selecting, modifying, and accepting information needed for identifying a cardiac event for a subject in accordance with the methods disclosed herein. The workstation is further enabled to display the image frames 703 comprising the video. In other embodiments, a user or technician uses the workstation to view clusters, generate clusters, label or re-label clusters, assign or re-assign cardiac signal segments to clusters, identify features of interest, associate various features of interest with different cardiac events, assign or re-assign cardiac events to different clusters, set various parameters, select or otherwise define segments of cardiac signals for processing, and use the workstation to facilitate the functionality of any of the modules of system 700. User input and user selections may be stored/retrieved in any of the storage devices 713 and 726. Default settings and initial parameters can be retrieved from any of the storage devices. A user may adjust various parameters being utilized or dynamically adjust settings in real-time during processing. The alert signal generated by module 717 may be received and viewed by the workstation and/or communicated to one or more remote devices over network 727.

Although shown as a desktop computer, it should be appreciated that the workstation can be a laptop, mainframe, or a special purpose computer such as an ASIC, circuit, or the like. The embodiment of the workstation is illustrative and may include other functionality known in the arts. Any of the components of the workstation may be placed in communication with any of the modules of system 700 or any devices placed in communication therewith. Moreover, any of the modules of system 700 can be placed in communication with storage device 726 and/or computer readable media 722 and may store/retrieve therefrom data, variables, records, parameters, functions, and/or machine readable/executable program instructions, as needed to perform their intended functions. Any of the modules of system 700 may be placed in communication with one or more remote devices over network 727. It should be appreciated that some or all of the functionality performed by any of the modules or processing units of system 700 can be performed, in whole or in part, by the workstation. The embodiment shown is illustrative and should not be viewed as limiting the scope of the appended claims strictly to that configuration. Various modules may designate one or more components which may, in turn, comprise software and/or hardware designed to perform the intended function.

### Performance Results

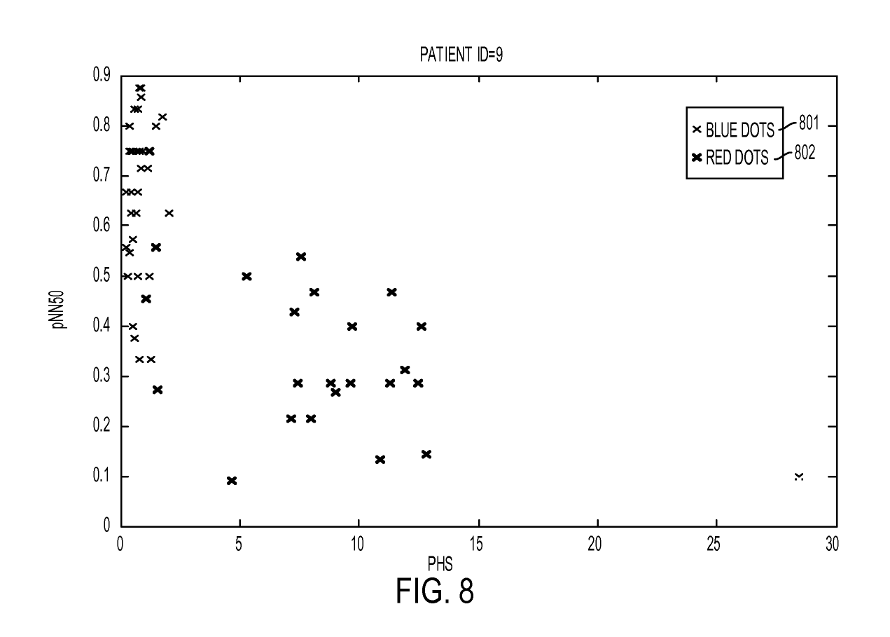

In FIG. 8, two metrics are plotted, i.e., PHS and pNN50, calculated before/after the patient went through a cardioversion procedure. Dots 801 and 802 were from AF (before) and SR (after) segments, respectively. The segments were manually ground truthed by trained professionals. To further separate the 2 classes, various transformations can be employed.

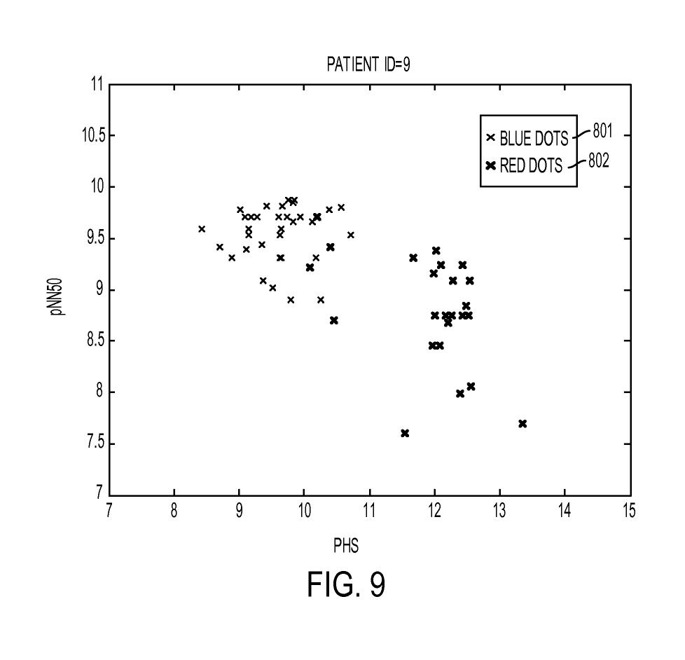

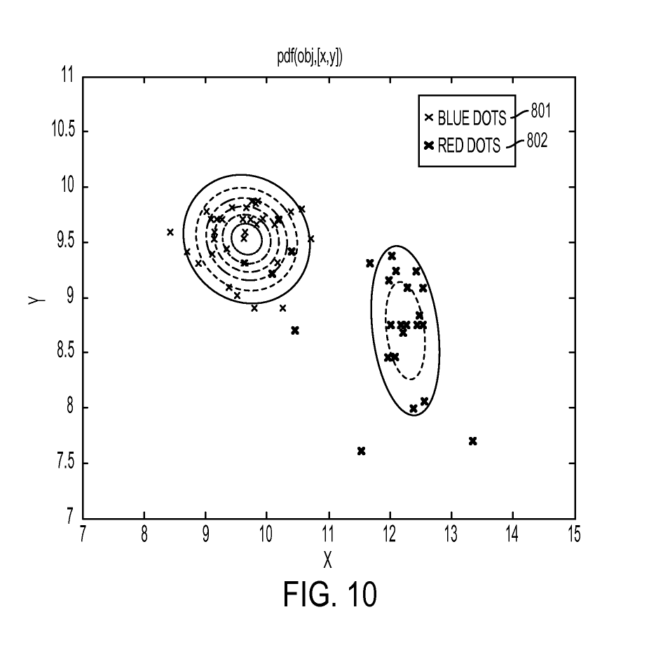

FIG. 9 shows an example using a log-transform of our raw data. A bi-modal Gaussian-Mixture Model (GMM) was estimated from this data set and is shown in FIG. 10. As can be seen from FIG. 10, there are two clusters formed by the GMM. The log-transformation is just one example from many different choices of transformations. Although the GMM used only 2 metrics, additional metrics can be utilized. In FIG. 8, a bi-modal GMM was constructed because of prior knowledge of the data which was taken in multiple 15-second segments both before AF and after SR. In cases which we can only observe the patient either before or after the cardioversion procedure, a unimodal distribution for the specific state can be constructed. Furthermore, in cases which we are not sure if the data consists of only one of the two states (before vs. after) or a mixture of both states, we can fit the data with both unimodal and bimodal distributions and use Akaike Information Criterion (AIC) or Bayes Information Criterion (BIC) to select the better performing model. Both AIC and BIC are negative log-likelihoods for the data with penalty terms for the number of estimated parameters. Both can be used to determine an appropriate number of components for a model when the number of components is unspecified. Once the model is obtained, for any new data points received, one or a set of statistical measures (e.g., Mahalanobis distance) or hypothesis test (e.g., χ² test) are calculated to determine the likelihood of the new data point coming from the particular distribution (AF or SR). This approach allows for the detection of the presence of AF with very limited data points for early detection of reoccurrence of AF after a cardioversion procedure. It doesn't require manually labeling of ground truth of each segment for a training set. The new data points can also be easily incorporated into the model by recalculating the model parameters such as mean and standard deviation for a normal distribution.

We used the Mahalanobis distance to the cluster centers to identify AF by selecting the shorter distance to the cluster centers. Alternatively, a predetermined threshold can be utilized. For example, for the patient data shown in FIG. 8, the GMM model was constructed using all of the data points except leaving one from before or one from after cardioversion (i.e., the leave one out cross-validation (LOOCV) technique). The model parameters are shown here as:
Gaussian mixture distribution with 1 component in 2 dimensions
Component 1:
Mixing proportion: 1.000000
Mean: 10.5441 9.2350
   and
Gaussian mixture distribution with 2 components in 2 dimensions
Component 1:
Mixing proportion: 0.660841
Mean: 9.6757 9.5137
Component 2:
Mixing proportion: 0.339159
Mean: 12.2362 8.6922

The 2^{nd} model was selected based on having a lower AIC value between the 2 models.

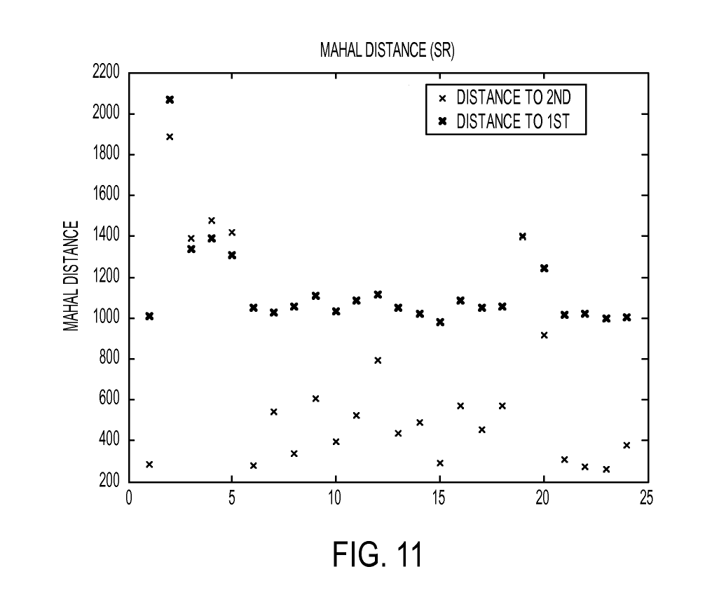

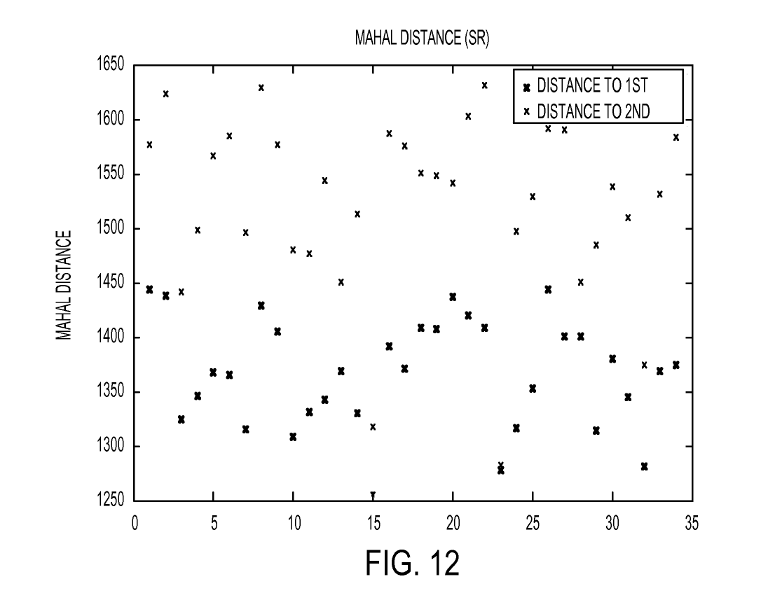

The Mahalanobis distances to the 2 cluster centers were then calculated and the shorter distance between the 2 was used to identify each segment as AF or SR. FIGS. 11 and 12 show the distance distributions for segments taken before and after the cardioversion procedure, respectively. The error rate is about 10% with all of them as FP's (SR classified as AF). The same approach was repeated for 3 more patients using more feature metrics with an error rate of about 15%.

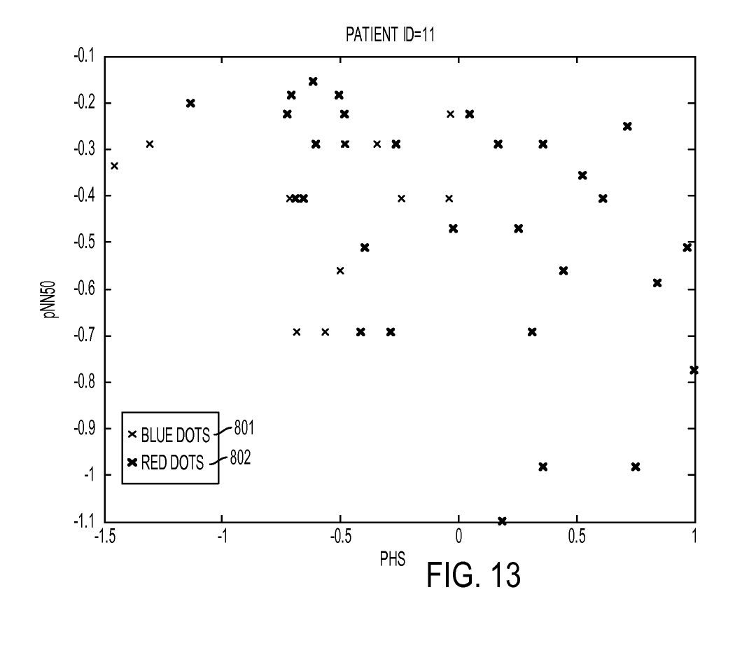

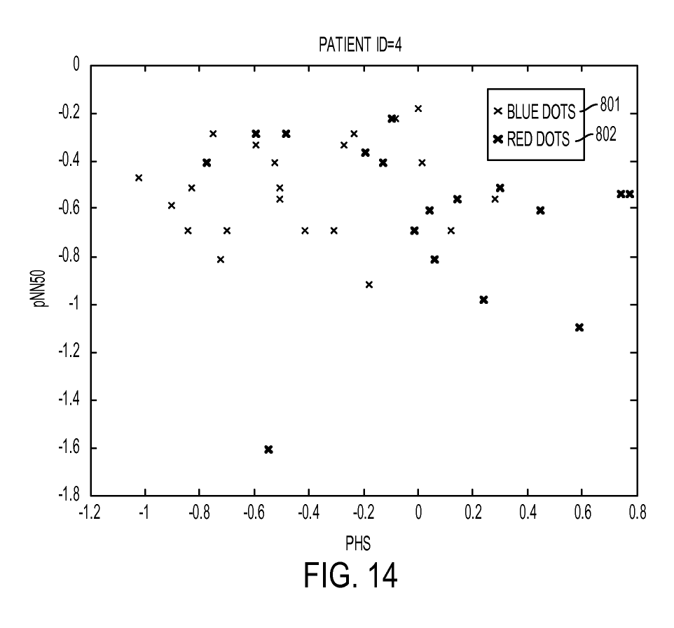

It should be noted that not all patients who went through the cardioversion procedure gave the same error rate. For example, for 2 of our patients, there wasn't enough distinction between the 2 states. For the two patients in FIGS. 13 and 14, only one cluster (2 identical clusters) was identified. Since we know the data consists of both AF and SR, obviously this model is not 100% correct. One possible cause was the limited number of samples from these 2 patients (e.g., FIG. 13 had only 38 data points in total). When we use ½ of all available segments from all patients to construct the model and the other ½ for testing, the error rate was 20% overall with about a 70% true-positive rate and about a 20% false-positive rate. Another cause is the noise in extracting the metrics, e.g., peak detection which leads to noise in 6 out of the 7 metrics. In the entire data set, there were about 19% error rate in peak detection alone (approximately 1% for over-detect and approximately 18% for misdetection). Here, our model parameters were as follows:
Gaussian mixture distribution with 2 components in 2 dimensions
Component 1:
Mixing proportion: 0.493615
Mean: 9.7546 9.4401
Component 2:
Mixing proportion: 0.506385
Mean: 9.7546 9.4401

Even though the present method is not perfect for all patients in the patient group we analyzed, improvement is anticipated by, for example, increasing the number of samples (from each patient or patient group) and exploring more features of interest. However, it is clear from our tests that a personalized model can be constructed for individuals and for groups of individuals which provides benefits such as: (1) it doesn't need to be constrained to one particular feature; (2) the construction of the model doesn't require the labeling of ground truth of each signal segment; and (3) it doesn't require the presence of both AF and SR for cluster formation purposes. If the ground truth of a set of signal segments is known, another approach is to train a classifier such as SVM or others such as a Neural Network or discriminant-based classifiers. The classifier can be trained for each individual patient or a patient pool.

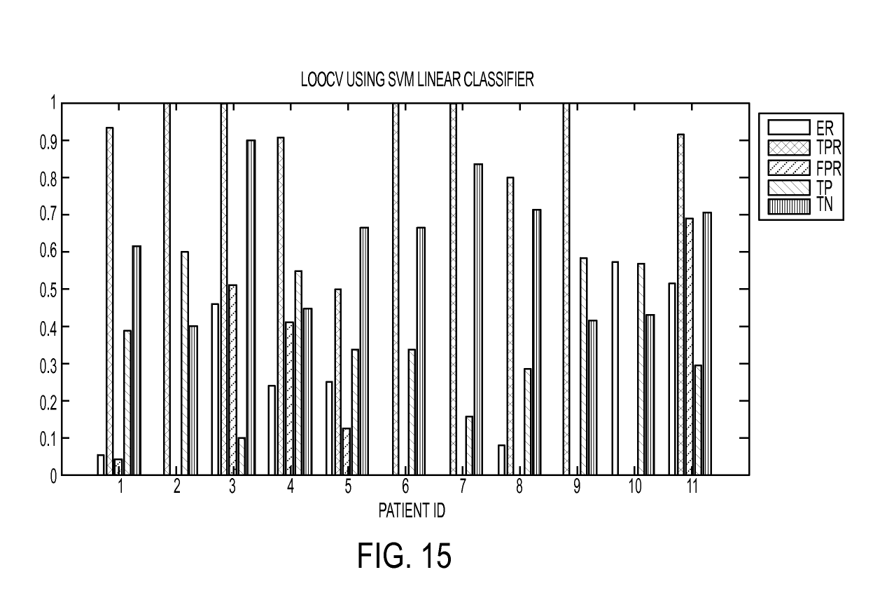

FIG. 15 shows the classification results for 11 patients using a trained SVM classifier in a LOOCV experiment. The weighted average error rate is about 17%.

Overall, the present method is adaptive because it can use any of a wide array of features which have been identified as being of interest for detection of various cardiac events. The construction of the model doesn't require the labeling of ground truth of each signal segment.

### Various Embodiments

The teachings hereof can be implemented in hardware or software using any known or later developed systems, structures, devices, and/or software by those skilled in the applicable art without undue experimentation from the functional description provided herein with a general knowledge of the relevant arts. One or more aspects of the methods described herein are intended to be incorporated in an article of manufacture. The article of manufacture may be shipped, sold, leased, or otherwise provided separately either alone or as part of a product suite or a service.

The above-disclosed and other features and functions, or alternatives thereof, may be desirably combined into other different systems or applications. Presently unforeseen or unanticipated alternatives, modifications, variations, or improvements may become apparent and/or subsequently made by those skilled in this art which are also intended to be encompassed by the following claims. The teachings of any publications referenced herein are hereby incorporated by reference in their entirety.

What is claimed is:

1. A method for identifying a type of cardiac event from a cardiac signal obtained from a subject, the method comprising:
forming at least two clusters containing elements comprising at least cardiac signal segments which have been assigned to said clusters based on features of interest obtained from each respective signal segment, each of said clusters being associated with a different cardiac event;
receiving a new cardiac signal segment of a subject which has not yet been assigned to one of said clusters;
analyzing said new cardiac signal segment to obtain at least one feature of interest; and
identifying a type of cardiac event for said subject based on the cardiac event associated with one of said clusters which said feature of interest obtained from said subject's cardiac signal segment had a shortest distance to.

2. The method of claim 1, wherein said cardiac signal segments have been assigned to one of said clusters based on any of: a manual assignment, and an automatic assignment based on a distance between signal segments.

3. The method of claim 1, wherein said feature of interest is any of: a cardiac signal, a frequency domain version of said cardiac signal segment, higher order statistical quantities of said cardiac signal segment comprising any of: a mean, standard deviation, skew, and kurtosis of a set of peak-to-peak intervals of said cardiac signal segment, and a heart rate variability metric comprising any of: Standard Deviation of RR Intervals (SDRR), Root Mean Square of Successive RR Difference (RMSSD), Proportion of NN or RR interval exceeding 50 milliseconds (pNN50), Shannon Entropy (ShE), Standard Deviation 1 (SD1), Standard Deviation 2 (SD2), Pulse Harmonic Strength (PHS) and a Normalized Pulse Harmonic Strength (NPHS).

4. The method of claim 1, further comprising labeling said clusters by a type of cardiac event based on any of: electrocardiographic traces, manual labeling, apriori knowledge of different cardiac events, and a heart rate variability metric for said cardiac signal segment comprising any of: Standard Deviation of RR Intervals (SDRR), Root Mean Square of Successive RR Difference (RMSSD), Proportion of NN or RR interval exceeding 50 milliseconds (pNN50), Shannon Entropy (ShE), Standard Deviation 1 (SD1), Standard Deviation 2 (SD2), Pulse Harmonic Strength (PHS) and a Normalized Pulse Harmonic Strength (NPHS).

5. The method of claim 1, wherein said shortest distance between said feature of interest and one of said clusters is determined in relation to any of: a center of said cluster, a boundary element of said cluster, and a weighted sum of at least some elements in said cluster, said distance being any of: Euclidean, Mahalanobis, Bhattacharyya, Hamming, and a Hellinger distance.

6. The method of claim 1, wherein forming said clusters comprises performing on said feature of interest at least one of: K-means testing, vector quantization, constrained clustering, fuzzy clustering, linear discriminant analysis, Gaussian Mixture Model, nearest neighbor clustering, manual sorting, and a support vector machine.

7. The method of claim 1, wherein at least one of said clusters is associated with a type of cardiac event which is an arrhythmic event comprising any of: atrial fibrillation, ventricular premature contraction, ventricular tachycardia, sinus bradycardia, and sinus tachycardia.

8. The method of claim 1, wherein at least one of said clusters is associated with a cardiac event which is a non-arrhythmic event.

9. The method of claim 1, wherein any of said cardiac signal segments is one of: an electrocardiographic signal from an electrocardiographic device, a ballistocardiographic signal from a ballistocardiographic device, an electroencephalographic signal from an electroencephalographic device, an echocardiographic signal from an echocardiographic device, an electromyographic signal from an electromyographic device, a phonocardiographic signal from a phonocardiographic device, and a videoplethysmographic signal from a video imaging device comprising any of: a contact-based video camera, a non-contact-based video camera, a RGB camera, a multi-spectral camera, a hyperspectral camera, and a hybrid camera comprising any combination hereof.

10. The method of claim 1, wherein said cardiac signal segment is normalized to a frequency of a normalized heartbeat.

11. The method of claim 1, wherein a length of said cardiac signal segment is one of: a single cardiac cycle, and a normalized cardiac cycle.

12. The method of claim 1, wherein, in response to having identified said subject's type of cardiac event, further comprising any of: initiating an alert, and signaling a medical professional.

13. The method of claim 1, further comprising communicating said identified cardiac event to any of: a memory, a storage device, a display device, a handheld wireless device, a handheld cellular device, and a remote device over a network.

14. A system for identifying a type of cardiac event from a cardiac signal obtained from a subject, the system comprising:
a storage device storing at least two clusters containing elements comprising at least cardiac signal segments which have been assigned to said clusters based on features of interest obtained from each respective signal segment, each of said clusters being associated with a different cardiac event; and
a processor in communication with a memory and said storage device, said processor executing machine readable instructions for performing:
receiving a new cardiac signal segment of a subject which has not yet been assigned to one of said clusters;
analyzing said new cardiac signal segment to obtain at least one feature of interest; and
identifying a type of cardiac event for said subject based on the cardiac event associated with one of said clusters which said feature of interest obtained from said subject's cardiac signal segment had a shortest distance to.

15. The system of claim 14, wherein said cardiac signal segments have been assigned to one of said clusters based on any of: a manual assignment, and an automatic assignment based on a distance between signal segments.

16. The system of claim 14, wherein said feature of interest is any of: a cardiac signal, a frequency domain version of said cardiac signal segment, higher order statistical quantities of said cardiac signal segment comprising any of: a mean, standard deviation, skew, and kurtosis of a set of peak-to-peak intervals of said cardiac signal segment, and a heart rate variability metric comprising any of: Standard Deviation of RR Intervals (SDRR), Root Mean Square of Successive RR Difference (RMSSD), Proportion of NN or RR interval exceeding 50 milliseconds (pNN50), Shannon Entropy (ShE), Standard Deviation 1 (SD1), Standard Deviation 2 (SD2), Pulse Harmonic Strength (PHS) and a Normalized Pulse Harmonic Strength (NPHS).

17. The system of claim 14, further comprising labeling said clusters by a type of cardiac event based on any of: electrocardiographic traces, manual labeling, apriori knowledge of different cardiac events, and a heart rate variability metric for said cardiac signal segment comprising any of: Standard Deviation of RR Intervals (SDRR), Root Mean Square of Successive RR Difference (RMSSD), Proportion of NN or RR interval exceeding 50 milliseconds (pNN50), Shannon Entropy (ShE), Standard Deviation 1 (SD1), Standard Deviation 2 (SD2), Pulse Harmonic Strength (PHS) and a Normalized Pulse Harmonic Strength (NPHS).

18. The system of claim 14, wherein said shortest distance between said feature of interest and one of said clusters is determined in relation to any of: a center of said cluster, a boundary element of said cluster, and a weighted sum of at least some elements in said cluster, said distance being any of: Euclidean, Mahalanobis, Bhattacharyya, Hamming, and a Hellinger distance.

19. The system of claim 14, wherein forming said clusters comprises performing on said feature of interest at least one of: K-means testing, vector quantization, constrained clustering, fuzzy clustering, linear discriminant analysis, Gaussian Mixture Model, nearest neighbor clustering, manual sorting, and a support vector machine.

20. The system of claim 14, wherein at least one of said clusters is associated with a type of cardiac event which is an arrhythmic event comprising any of: atrial fibrillation, ventricular premature contraction, ventricular tachycardia, sinus bradycardia, and sinus tachycardia.

21. The system of claim 14, wherein at least one of said clusters is associated with a cardiac event which is a non-arrhythmic event.

22. The system of claim 14, wherein any of said cardiac signal segments is one of: an electrocardiographic signal from an electrocardiographic device, a ballistocardiographic signal from a ballistocardiographic device, an electroencephalographic signal from an electroencephalographic device, an echocardiographic signal from an echocardiographic device, an electromyographic signal from an electromyographic device, a phonocardiographic signal from a phonocardiographic device, and a videoplethysmographic signal from a video imaging device comprising any of: a contact-based video camera, a non-contact-based video camera, a RGB camera, a multi-spectral camera, a hyperspectral camera, and a hybrid camera comprising any combination hereof.

23. The system of claim 14, wherein said cardiac signal segment is normalized to a frequency of a normalized heartbeat.

24. The system of claim 14, wherein a length of said cardiac signal segment is one of: a single cardiac cycle, and a normalized cardiac cycle.

25. The system of claim 14, wherein, in response to having identified said subject's type of cardiac event, further comprising any of: initiating an alert, and signaling a medical professional.
